SEQUENCE LISTING (7 sequences)

SEQ ID NO: 1 - DNA, 20 bp, Artificial Sequence
  Synthetic Primer
ccatgccagagaatggatct

SEQ ID NO: 2 - DNA, 20 bp, Artificial Sequence
  Synthetic Primer
attcaggtctgttccgatgc

SEQ ID NO: 3 - DNA, 20 bp, Artificial Sequence
  Synthetic Primer
tagcacagcctggatagcaa

SEQ ID NO: 4 - DNA, 21 bp, Artificial Sequence
  Synthetic Primer
caggcagtataatccaaagat

SEQ ID NO: 5 - DNA, 22 bp, Artificial Sequence
  Synthetic Primer
tctggcttatatccaacacttc

SEQ ID NO: 6 - DNA, 16 bp, Artificial Sequence
  Synthetic Primer
gccgtgtgtgtacctg

SEQ ID NO: 7 - DNA, 16 bp, Artificial Sequence
  Synthetic Primer
aaaggtgcgtcaagtt

(19) United States
(12) Patent Application Publication
(10) Pub. No.: US 2013/0052186 A1
(43) Pub. Date: Feb. 28, 2013

(54) Plasma Carboxypeptidase B as a Predictor for Disease Severity and Response

(71) Applicants: Jungsik Song, Stanford, CA (US); William H. Robinson, Palo Alto, CA (US)

(72) Inventors: Jungsik Song, Stanford, CA (US); William H. Robinson, Palo Alto, CA (US)

(21) Appl. No.: 13/492,099

(22) Filed: Jun. 8, 2012

Related U.S. Application Data
(60) Provisional application No. 61/494,763, filed on Jun. 8, 2011.

(51) Int. Cl. G01N 33/566; A61K 39/395; C40B 40/06; C12Q 1/68; C40B 30/04

(52) U.S. Cl. 424/130.1; 435/6.11; 435/6.12; 435/7.92; 436/501; 435/7.1; 506/9; 506/16

(57) ABSTRACT

Compositions and methods are provided for prognostic classification of individuals into groups that are informative of the individual's likelihood of developing severe disease associated with undesirable complement activation. Individuals having one or both alleles for a more stable or active carboxypeptidase B variant have a reduced propensity for developing severe disease. The presence of the protective variant may be identified through any suitable method.

### FEDERALLY SPONSORED RESEARCH AND DEVELOPMENT

This invention was made with Government support under grant no. N01-HV-28183 awarded by the National Institutes of Health. The Government has certain rights in this invention.

### BACKGROUND OF THE INVENTION

Rheumatoid arthritis (RA) is a painful and debilitating joint disease that affects up to 1% of the population (around 1.3 million adults in 2005 in the United States alone). It usually strikes after the age of 40, and is at least twice as common in women as in men. According to the CDC, RA patients often suffer reduced quality of life, as they experience pain and loss of function, affecting their work, leisure and social interactions. RA is a chronic disease, and may therefore continue indefinitely with frequent flare-ups, particularly in patients with a more severe form of the disease. Approximately 10% of patients with RA are severely disabled.

Inflammation associated with RA is characterized by activation of both inflammatory and coagulation pathways. Fibrin deposition, the culmination of the coagulation cascade, is a hallmark of RA synovium. Deposited fibrin can promote inflammatory responses, while citrulline-modified fibrinogen is a prominent target of RA-specific autoantibodies.

Improved methods for prediction of disease severity and response to therapy in RA and other complement associated conditions are of particular interest for clinical and other studies. The present invention addresses this issue.

### SUMMARY OF THE INVENTION

Compositions and methods are provided for prognostic classification of individuals into groups that are informative of the individual's likelihood of developing severe disease associated with undesirable complement activation, which diseases include without limitation rheumatoid arthritis, paroxysmal nocturnal hemoglobinuria (PNH), neuromyelitis optica (NMO). Individuals having one or both alleles for a more stable plasma carboxypeptidase B (CPB) variant have a reduced propensity for developing severe disease.

Exemplary of such protective alleles is the carboxypeptidase B variant Thr325Ile, which has increased stability under physiological conditions. The presence of the variant may be identified through any suitable method, including for example genotyping, e.g. determining the presence of the rs1926447 [C1040T encoding Thr325Ile] allele or the closely linked minor allele of CPB SNP rs1409433; direct sequencing of the CPB coding sequence; protein characterization, and the like. It is shown herein that CPB Thr325Ile decreases C5 activity, including activity in synovial joints, and is protective for disease progression.

Assessment in a patient allows improved care, where patients classified according to responsiveness can be treated with an appropriate agent. Individuals that lack the protective CPB allele, (in other words individuals that are homozygous for CPB 325Thr, coding sequence 1064C), have a high probability of responsiveness to a complement inhibitor therapy, including therapy that inhibits complement C5. Suitable therapies include, without limitation, antibodies that inhibit C5 activity, e.g. pexelizumab, eculizumab, etc. Patients can be classified upon initial presentation of symptoms, and can be further monitored for status over the course of the disease to maintain appropriate therapy, or can be classified at any appropriate stage of disease progression. In an embodiment, the method further comprises selecting a therapeutic regimen based on the analysis. In an embodiment, the method further comprises determining a treatment course for the subject based on the analysis. In an embodiment, the method further comprises assessing a clinical factor, e.g. determination of the presence of disease such as RA, NO, etc. in the mammalian subject; and combining the assessment with the analysis of the marker to the assessment of the prognosis for responsiveness of the subject to the therapy of interest.

In other embodiments of the invention a device or kit is provided for the analysis of patient samples. Such devices or kits will include reagents that specifically identify the presence of the CPB variant protein Thr325Ile, or genetic sequences that encode the variant, or genetic markers that are linked to the variant. Devices of interest include arrays, where the reagents are spatially separated on a substrate such as a slide, gel, multi-well plate, etc. Alternatively the reagents can be provided as a kit comprising reagents in a suspension or suspendable form, e.g. reagents bound to beads suitable for flow cytometry, and the like.

In some embodiments, a method is provided for treating complement mediated inflammatory diseases in a subject, the method comprising classifying the individual with respect to CPB, and in individuals that lack the protective variant CPB Thr325Ile, administering to the individual an effective dose of a complement inhibitor, e.g. a C5 complement inhibitor, and/or administering an effective amount of the protective variant of carboxypeptidase B, e.g. by providing a nucleic acid that encodes carboxypeptidase B operably linked to a promoter, administering active protein, etc. The therapeutic agent may be administered systemically, e.g. i.v., or locally, e.g. to the site of inflammatory lesions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. CPB protects against, whereas C5 promotes, inflammatory arthritis. Anti-collagen antibody-induced arthritis (CAIA) was generated by intravenous injection of anti-collagen antibodies on day 0, followed by intraperitoneal injection of lipopolysaccharide on day 3. (a) CAIA severity and paw thickness in Cpb2^{-/-} and Cpb2^{+/+} mice. Compared to controls, Cpb2^{-/-} mice exhibited significantly more severe arthritis from day 7 onwards. (b) Histological scoring of arthritis severity based on degree of inflammation, synovial hyperplasia, and bone or cartilage erosions in the mice used in (a). (c) Representative H&E-stained sections of joint tissue from mice used in (a). Arrowhead=inflammatory cell infiltrates, black arrow=erosions of bone or cartilage, white arrow=synovial hyperplasia. (d) Gene-dose effect of Cpb2 on CAIA. CAIA severity was measured in Cpb2^{+/+}, Cpb2^{+/-}, and Cpb2^{-/-} mice. One copy of Cpb2 provides the same level of protection against CAIA as do two copies. (e) CAIA severity in C5-deficient mice, OPN-deficient mice (Spp1^{-/-}), and bradykinin receptor 2-deficient mice (Bdkrb2^{-/-}) and their respective controls. C5 deficiency confers protection against CAIA. (f) CAIA severity in Cpb2^{-/-} and Cpb2^{+/+} mice treated with tranexamic acid (TA) starting one day before the induction of CAIA. TA aggravates CAIA in Cpb2^{-/-} mice. Results are the mean±s.e.m. and are representative of 2-3 independent experiments (n=4-5 per experimental group). ** P<0.01 and * P<0.05 by Mann-Whitney U test as compared with control mice at each time point.

FIG. 2. CPB cleavage of C5a suppresses neutrophil and macrophage chemotaxis to the peritoneum and synovium. (a) MALDI-TOF analysis of C5a incubated with PBS or CPB, showing the loss of arginine from CPB-treated C5a (bottom panel). (b) Flow-cytometric analysis of peritoneal-fluid cells from B6 mice injected intraperitoneally (i.p.) with CPB-treated or PBS-treated C5a or with CPB alone. Antibodies against the neutrophil marker Gr1 were used to identify neutrophils. (c) H&E-stained sections of mouse stifle joints injected with CPB-treated or PBS-treated C5a or with PBS alone. Arrows show inflammatory cell infiltrates in synovial tissues; there is less accumulation of immune cells in response to CPB-cleaved C5a than PBS-treated C5a. (d) Flow-cytometric analysis of peritoneal-fluid cells from Cpb2^{-/-} and Cpb2^{+/+} mice injected i.p. with Zymosan A (ZyA). Antibodies against F4/80 (macrophage marker) and Gr1 (neutrophil marker) were used. Results are the mean±s.e.m. and are representative of 2 independent experiments (n=5 per experimental group; ** P<0.01, * P<0.05 by one-way ANOVA and Tukey's test).

FIG. 3. RA patients who possess the CPB2 1040T allele, encoding long-half-life CPB, have a lower risk of progressing to radiographically severe RA. (a) Percentage of RA patients in the CLEAR I cohort (n=118) who developed radiographically severe RA (defined as the top tertile of radiographic severity based on modified Sharp/van der Heijde Score (SHS)) stratified by the nonsynonymous CPB2 SNPs rs1926447 (C1040T) and rs3742264 (G505A). Patients were divided into 2 groups, one containing 1040C homozygotes (CC) and one containing 1040T carriers (CT or TT). Compared with 1040C homozygotes, fewer carriers of 1040T, which encodes long-half-life, Ile325 CPB, developed radiographically severe RA within 3 years (39% 1040C homozygotes versus 13% 1040T carriers; P=0.026 by chi-square test). In contrast, the G505A (rs3742264) genotype was not associated with radiographic severity (N.S.=not significant). (b) C5a-desArg generation by CPB variants. The ratio of C5a-desArg to intact C5a at each time point was measured by mass spectrometry, the data fit was calculated by one-phase exponential models, and the half-times (50% of the time necessary to reach the calculated plateau) of C5a cleavage determined. The Ile325 CPB variant half-time was twice as long as that of Thr325 CPB. (c) C5a neutralization by CPB variants. Long-half-life Ile325 CPB is more efficient than short-half-life Thr325 CPB at neutralizing C5a activity, as assessed by C5a-mediated induction of neutrophil myeloperoxidase (MPO) release (** P<0.01; Student's t-test). Activity of C5a over time is presented as a percentage of the activity of C5a at 0 min of treatment with CPB.

FIG. 4. Characterization of CPB in RA joints. (a) CPB and total C5a levels are elevated in RA (n=20) compared to OA (n=19) synovial fluid. CPB levels are correlated with total C5a levels (P<0.01 by Pearson's correlation test) in RA and OA synovial fluid. (b) CPB2 mRNA expression in cells derived from RA synovial fluid and tissue. Liver cDNA was used as a positive control. (c) CPB2 mRNA is expressed in macrophages derived from a healthy donor. CPB2 cDNA was used as a positive control. (d) CPB2 mRNA expression in the promonocytic cell line U937 is increased following treatment with dexamethasone or M-CSF. (e) Immunoblot analysis of proCPB expression in U937 cells shows dose-dependent induction of CPB expression by dexamethasone. Control cell lysates were from the Huh-7.5 hepatocyte cell line. Results are the mean±s.e.m of triplicates and are representative 2 independent experiments. (**P<0.01, *P<0.05 by one-way ANOVA and Dunnett's test).

FIG. 5. Similar levels of CPB are present in plasma derived from RA patients (n=10) and healthy controls (n=20). N.S.=not significant.

FIG. 6. Immunohistochemical detection of CPB in RA synovium. Sections of paraff in-embedded RA synovium were stained with a rabbit polyclonal anti-CPB antibody (left panels). Brown color indicates positive staining. Staining of RA synovial tissue demonstrates that CPB is predominantly detected in an interstitial distribution, but that there are also cells (arrows) that exhibited a cytoplasmic staining pattern suggestive of local production of CPB in RA synovium. Rabbit immunoglobulin G was used as a negative isotype control (right panels).

### DETAILED DESCRIPTION

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Polymorphism, as used herein refers to variants in the gene sequence. Such variants may include single nucleotide polymorphisms, splice variants, insertions, deletions and transpositions. The polymorphisms can be those variations (DNA sequence differences) that are generally found between individuals or different ethnic groups and geographic locations which, while having a different sequence, produce functionally equivalent gene products. Polymorphisms also encompass variations which can be classified as alleles and/or mutations which can produce gene products which may have an altered function, i.e. variants in the sequence which can lead to gene products that are not functionally equivalent. Polymorphisms also encompass variations which can be classified as alleles and/or mutations which either produce no gene product, an inactive gene product or increased gene product. Further, the term is also used interchangeably with allele as appropriate.

Where a polymorphic site is a single nucleotide in length, the site is referred to as a single nucleotide polymorphism (“SNP”).

As used herein, the term “nucleic acid probe” refers to a molecule capable of sequence specific hybridization to a nucleic acid, and includes analogs of nucleic acids, as are known in the art, e.g. DNA, RNA, peptide nucleic acids, and the like, and may be double-stranded or single-stranded. Alterations of the nucleic acid molecules can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

“Specific hybridization,” as used herein, refers to the ability of a first nucleic acid to hybridize to a second nucleic acid in a manner such that the first nucleic acid does not hybridize to any nucleic acid other than to the second nucleic acid. “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions ×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., W=5 or W=20).

Nucleic acid molecules of interest as probes are at least about 15, preferably at least about 18, 20, 23 or 25 nucleotides, and can be 30, 40, 50, 100, 200 or more nucleotides in length, and encompass the polymorphic residues of the SNP markers described herein. A probe or primer comprises a region of nucleotide sequence that hybridizes to at least about 15, for example about 20-25, and in certain aspects about 40, 50 or 75 consecutive nucleotides of a nucleic acid molecule comprising a contiguous nucleotide sequence of CPB2 or polymorphic variants thereof. In other aspects, a probe or primer comprises 100 or fewer nucleotides, in certain aspects from 6 to 50 nucleotides, for example from 12 to 30 nucleotides. In other aspects, the probe or primer is at least 70% identical to the contiguous nucleotide sequence or to the complement of the contiguous nucleotide sequence, for example at least 80% identical, in certain aspects at least 90% identical, and in other aspects at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

A “marker”, as described herein, refers to a genomic sequence characteristic of a particular allele at a polymorphic site.

SNP nomenclature as reported herein refers to the official Reference SNP (rs) ID identification tag as assigned to each unique SNP by the National Center for Biotechnological Information (NCBI).

A “haplotype,” as described herein, refers to a segment of a genomic DNA strand that is characterized by a specific combination of genetic markers (“alleles”) arranged along the segment. In a certain embodiment, the haplotype can comprise one or more alleles, two or more alleles, three or more alleles, four or more alleles, or five or more alleles.

The term “susceptibility”, as described herein, means primarily increased susceptibility. Thus, particular markers and/or haplotypes of the invention may be characteristic of increased susceptibility to severe autoimmune disease, e.g. RA, NO, etc., as characterized by a relative risk of greater than one. Markers and/or haplotypes that confer increased susceptibility of severe disease are furthermore considered to be “at-risk”, as they confer an increased risk of disease.

“Carboxypeptidase B” shall mean one of the variant human proteins identified as CPB2, i.e. the protein sequences set forth in Genbank NP_{—}057497.3 and NP_{—}001863.2, which are encoded by the mRNA sequences set forth in GenBank Accession No. NM_{—}016413.3 and NM_{—}001872.3, respectively. Active fragments of carboxypeptidase B share a functional or binding property with full length Carboxypeptidase B. Epitopic fragments of carboxypeptidase B bind to a monoclonal antibody that binds to full length Carboxypeptidase B.

“Activity” of carboxypeptidase B shall mean any enzymatic or binding function performed by that protein, particularly the cleavage of complement in a blood sample or derivative thereof.

“Comparable cell” shall mean a cell whose type is identical to that of another cell to which it is compared. Examples of comparable cells are cells from the same cell line.

“Expressible nucleic acid” shall mean a nucleic acid encoding a nucleic acid of interest and/or a protein of interest, which nucleic acid is an expression vector, plasmid or other construct which, when placed in a cell, permits the expression of the nucleic acid or protein of interest. Expression vectors and plasmids are well known in the art.

“Inhibiting” the severity of a disorder shall mean lessening the likelihood of the disorder progressing to a serious form, or improving the prognosis of a serious form of the disorder. The methods of the invention are usually applied to patients that have been diagnosed with a disease associated with undesirable complement activation, which diseases include without limitation rheumatoid arthritis, paroxysmal nocturnal hemoglobinuria (PNH), neuromyelitis optica (NMO).

“Inhibiting” the expression of a gene in a cell shall mean either lessening the degree to which the gene is expressed, or preventing such expression entirely.

“Subject” or “patient” shall mean any animal, such as a human, non-human primate, mouse, rat, guinea pig or rabbit.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular (mediated by antigen-specific T cells or their secretion products) response. An “immunogen” is capable of inducing an immunological response against itself on administration to a mammal or due to autoimmune disease.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer primarily to treatment of pre-existing conditions, for example to lessen symptoms, to prevent a worsening of disease severity, etc. The treatment of ongoing disease, where the treatment stabilizes or improves the clinical symptoms of the patient, is of particular interest.

### Conditions for Analysis and Therapy

The invention provides methods for classification of complement associated diseases. The inhibition of complement in animal models has been shown to reduce laboratory measures of kidney disease, asthma, transplantation, multifocal motor neuropathy, myasthenia gravis, lupus, rheumatoid arthritis, paroxysmal nocturnal hemoglobinuria (PNH) and neuromyelitis optica (NMO).

Rheumatoid Arthritis is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Environmental factors may also play a role. Immunologic changes may be initiated by multiple factors. About 0.6% of all populations are affected, women two to three times more often than men. Onset may be at any age, most often between 25 and 50 yr.

Prominent immunologic abnormalities that may be important in pathogenesis include immune complexes found in joint fluid cells and in vasculitis. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; interleukin-1, which stimulates lymphocyte proliferation; and prostaglandins. The infiltrating cells, initially perivenular but later forming lymphoid follicles with germinal centers, synthesize interleukin-2, other cytokines, RF, and other immunoglobulins. Fibrin deposition, fibrosis, and necrosis also are present. Hyperplastic synovial tissue (pannus) may erode cartilage, subchondral bone, articular capsule, and ligaments. PMNs are not prominent in the synovium but often predominate in the synovial fluid.

Onset is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

Neuromyelitis optica (NMO), or Devic's disease, is an autoimmune, inflammatory disorder of the optic nerves and spinal cord. Although inflammation can affect the brain, the disorder is distinct from multiple sclerosis, having a different pattern of response to therapy, possibly a different pattern of autoantigens and involvement of different lymphocyte subsets.

The main symptoms of Devic's disease are loss of vision and spinal cord function. As for other etiologies of optic neuritis, the visual impairment usually manifests as decreased visual acuity, although visual field defects, or loss of color vision can occur in isolation or prior to formal loss of acuity. Spinal cord dysfunction can lead to muscle weakness, reduced sensation, or loss of bladder and bowel control. The damage in the spinal cord can range from inflammatory demyelination to necrotic damage of the white and grey matter. The inflammatory lesions in Devic's disease have been classified as type II lesions (complement mediated demyelinization), but they differ from MS pattern II lesions in their prominent perivascular distribution. Therefore, the pattern of inflammation is often quite distinct from that seen in MS.

Attacks are treated with short courses of high dosage intravenous corticosteroids such as methylprednisolone IV. When attacks progress or do not respond to corticosteroid treatment, plasmapheresis can be used. Commonly used immunosuppressant treatments include azathioprine (Imuran) plus prednisone, mycophenolate mofetil plus prednisone, Rituximab, Mitoxantrone, intravenous immunoglobulin (IVIG), and Cyclophosphamide. The monoclonal antibody rituximab is under study.

The disease can be monophasic, i.e. a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. On the other hand, patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike MS, Devic's disease rarely has a secondary progressive phase in which patients have increasing neurologic decline between attacks without remission. Instead, disabilities arise from the acute attacks.

Paroxysmal nocturnal hemoglobinuria is a rare disorder characterized by intravascular hemolysis and hemoglobinuria, the latter accentuated during sleep. Leukopenia, thrombocytopenia, and episodic crises are common. Diagnosis requires flow cytometry. Treatment is supportive. PNH is an acquired genetic mutation resulting in a membrane defect in stem cells and their progeny, including RBCs, WBCs, and platelets. It results in unusual sensitivity to normal C3 in the plasma, leading to ongoing intravascular hemolysis of RBCs and diminished marrow production of WBCs and platelets. The defect is a missing glycosyl-phosphatidyl-inositol anchor for membrane proteins caused by an abnormality of the PIG-A gene, which is located on the X chromosome. Protracted urinary Hb loss may result in iron deficiency. Patients are strongly predisposed to both venous and arterial thrombi, including the Budd-Chiari syndrome. Thrombi are commonly fatal. Some patients with PNH develop aplastic anemia, and some with aplastic anemia develop PNH. Crises may be precipitated by infection, iron use, vaccination, or menstruation. Abdominal and lumbar pain and symptoms of severe anemia may occur; gross hemoglobinuria and splenomegaly are common.

PNH is suspected in patients who have typical symptoms of anemia or unexplained normocytic anemia with intravascular hemolysis, particularly if leukopenia or thrombocytopenia is present. Historically, if PNH was suspected, the sugar-water test was usually the first test done; it relies on enhanced hemolysis of C3-dependent systems in isotonic solutions of low ionic strength, is simple to do, and is sensitive. However, the test is nonspecific; positive results require confirmation by further testing. The most sensitive and specific test is determination of the absence of specific RBC or WBC membrane proteins (CD59 and CD55) by flow cytometry. An alternative is the acid hemolysis test (Ham's test). Hemolysis usually occurs if blood is acidified with HCl, incubated for 1 h, and centrifuged. Bone marrow examination is not necessary but, if done to exclude other disorders, usually shows marrow hypoplasia. Gross hemoglobinuria is common during crises, and the urine may contain hemosiderin.

### Diagnostic Methods

Genetic variants in the CPB2 gene are shown to be markers for an individual's susceptibility to severe RA and other complement-associated conditions. In diagnostic methods of the invention, an individual may be evaluated for the presence of polymorphisms in CPB2. In some embodiments the polymorphism is a single nucleotide polymorphism (SNP). Genotyping of a non-synonymous SNP (C1040T) in the CPB-encoding gene (CPB2) revealed that RA patients who possess the 1040T allele (encoding Ile325 CPB, a variant with longer half-life) had a lower risk of developing radiographically severe RA. Compared to 1040C homozygotes, carriers of the 1040T allele had a relative risk reduction of 70% for developing radiographically severe RA within 3 years. The long-half-life Ile325 CPB is three times as effective as the short-half-life Thr325 CPB at neutralizing the proinflammatory mediator C5a, a difference that may provide the molecular basis for the protective effect on the development of severe disease. Other SNPs, for example the closely linked minor allele of CPB SNP rs1409433, can also be diagnostic for the absence or presence of the protective allele.

Assessment of risk may include analysis of genomic regions contiguous with the genetic polymorphisms described herein. Large regions of DNA are often inherited as block and, as such, contain causal as well as non-causal polymorphisms in close proximity. Thus a polymorphism may be inherited as a “linkage disequilibrium block” or “LD block” together with numerous other polymorphisms.

In one embodiment of the invention, diagnosis of a susceptibility to severe disease is carried out by detecting a predisposing (or protective) polymorphism in CPB2. The polymorphism can be a change in a CPB2 nucleic acid, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; alternation is the splicing, or rearrangement of all or a part of the gene. More than one such change may be present in a single gene.

Such sequence changes can cause a difference in the polypeptide encoded by a CPB2 nucleic acid. For example, if the difference is a frame shift change, the frame shift can result in a change in the encoded amino acids, and/or can result in the generation of a premature stop codon, causing generation of a truncated polypeptide. Alternatively, a polymorphism associated with a disease or condition or a susceptibility to a disease or condition associated with a CPB2 nucleic acid can be a synonymous alteration in one or more nucleotides (i.e., an alteration that does not result in a change in the polypeptide encoded by a CPB2 nucleic acid). Such a polymorphism may alter splicing sites, affect the stability or transport of mRNA, or otherwise affect the transcription or translation of the gene. A CPB2 nucleic acid that has any of the changes or alterations described above is referred to herein as an “altered nucleic acid.”

Specific polymorphisms of interest include those present in an SNP of the CPB2 locus, e.g. rs1926447 and SNP rs1409433, as well as any one of the publicly known SNPs in the CPB2 locus that are closely linked to rs1926447.

For determining a susceptibility to development of severe disease, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual (RNA and cDNA can only be used for exonic markers), such as an individual suspected of having, being susceptible to or predisposed for, or carrying a defect for, severe RA. The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs.

The DNA, RNA, or cDNA sample is then examined to determine which allele of a polymorphism is present in a CPB2 nucleic acid. The presence of the allele of interest can be indicated by hybridization of the gene in the genomic DNA, RNA, or cDNA to a nucleic acid probe. A “nucleic acid probe”, as used herein, can be a DNA probe or an RNA probe; the nucleic acid probe can contain, for example, at least one polymorphic residue in a CPB2 nucleic acid. The probe can be any of the nucleic acid molecules described above (e.g., the gene or nucleic acid, a fragment, a vector comprising the gene or nucleic acid, a probe or primer, etc.)

A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA, and to distinguish between the risk allele and the protective allele.

The hybridization sample is maintained under conditions that are sufficient to allow specific hybridization of the nucleic acid probe to a CPB2 nucleic acid. “Specific hybridization”, as used herein, indicates exact hybridization (e.g., with no mismatches). Specific hybridization can be performed under high stringency conditions or moderate stringency conditions, for example, as described above. In a particularly preferred aspect, the hybridization conditions for specific hybridization are high stringency appropriate to the length of the probe.

Specific hybridization, if present, is then detected using standard methods. More than one nucleic acid probe can also be used concurrently in this method. Specific hybridization of any one of the nucleic acid probes to an allele indicated herein as being a predisposing allele is diagnostic for a susceptibility to severe RA.

In another method of the invention, alteration analysis by restriction digestion can be used to detect an alteration in the gene, if the alteration (mutation) or polymorphism in the gene results in the creation or elimination of a restriction site. A test sample containing genomic DNA is obtained from the individual. Polymerase chain reaction (PCR) can be used to amplify a CPB2 nucleic acid (and, if necessary, the flanking sequences) in the test sample of genomic DNA from the test individual. RFLP analysis is conducted as described (see Current Protocols in Molecular Biology). The digestion pattern of the relevant DNA fragment indicates the presence or absence of the allele indicated herein as being a predisposing allele in the CPB2 nucleic acid, and therefore indicates the presence or absence of a susceptibility to severe RA.

Sequence analysis can also be used to detect specific polymorphisms in a CPB2 nucleic acid. A test sample of DNA or RNA is obtained from the test individual. PCR or other appropriate methods can be used to amplify the gene or nucleic acid, and/or its flanking sequences, if desired. The sequence of a CPB2 nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA or mRNA, as appropriate. The presence of a polymorphism indicated herein as being a predisposing allele in the CPB2 gene indicates that the individual has a susceptibility to severe RA.

Allele-specific oligonucleotides can also be used to detect the presence of a polymorphism in a CPB2 nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs that specifically hybridizes to a CPB2 nucleic acid, and that contains a polymorphism associated with a susceptibility to severe disease. An allele-specific oligonucleotide probe that is specific for particular polymorphisms in a CPB2 nucleic acid can be prepared, using standard methods (see Current Protocols in Molecular Biology). The invention further provides allele-specific oligonucleotides that hybridize to the reference or variant allele of a gene or nucleic acid comprising a polymorphism or to the complement thereof. These oligonucleotides can be probes or primers. With the addition of such analogs as locked nucleic acids (LNAs), the size of primers and probes can be reduced to as few as 8 bases.

A test sample of DNA is obtained from the individual. PCR can be used to amplify all or a fragment of a CPB2 nucleic acid and its flanking sequences. The DNA containing the amplified CPB2 nucleic acid (or fragment of the gene or nucleic acid) is dot-blotted, using standard methods (see Current Protocols in Molecular Biology), and the blot is contacted with the respective oligonucleotide probe. The presence of specific hybridization of the probe to the amplified CPB2 nucleic acid is then detected. Hybridization of an allele-specific oligonucleotide probe to DNA from the individual is indicative of the presence of an allele indicated herein as being a predisposing allele in the CPB2 nucleic acid, and is therefore indicative of susceptibility to severe RA.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

In another aspect, arrays of oligonucleotide probes that are complementary to target nucleic acid sequence segments from an individual can be used to identify the presence of polymorphic alleles in a CPB2 nucleic acid. For example, in one aspect, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings are incorporated by reference herein. Techniques for the synthesis of these arrays using mechanical synthesis methods are described in, e.g., U.S. Pat. No. 5,384,261; the entire teachings are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphic alleles. Representative methods include direct manual sequencing (Church and Gilbert, Proc. Natl. Acad. Sci. USA 81:1991-1995 (1988); Sanger, F. et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one aspect of the invention, diagnosis of a susceptibility to development of severe disease can also be made by expression analysis by quantitative PCR (kinetic thermal cycling). This technique, utilizing TagMan® assays, can assess the presence of an alteration in the expression or composition of a CPB2 nucleic acid or splicing variants encoded by a CPB2 nucleic acid. TagMan® probes can also be used to allow the identification of polymorphisms and whether a patient is homozygous or heterozygous. Further, the expression of the variants can be quantified as physically or functionally different.

In another aspect of the invention, diagnosis of a susceptibility to severe disease can be made by examining expression and/or composition of a CPB2 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a CPB2 nucleic acid, or for the presence of a particular variant encoded by a CPB2 nucleic acid. An alteration in expression of a polypeptide encoded by a CPB2 nucleic acid can be, for example, an alteration in the quantitative polypeptide expression (i.e., the amount of polypeptide produced); an alteration in the composition of a polypeptide encoded by a CPB2 nucleic acid is an alteration in the qualitative polypeptide expression (e.g., expression of an altered CPB2 polypeptide).

Both such alterations (quantitative and qualitative) can also be present. The term “alteration” in the polypeptide expression or composition, as used herein, refers to an alteration in expression or composition in a test sample, as compared with the expression or composition of polypeptide by a CPB2 in a control sample. A control sample is a sample that corresponds to the test sample (e.g., is from the same type of cells), and is from an individual who is not affected by a susceptibility. An alteration in the expression or composition of the polypeptide in the test sample, as compared with the control sample, is indicative of a susceptibility to severe disease.

Various means of examining expression or composition of the polypeptide encoded by a CPB2 nucleic acid can be used, including: spectroscopy, colorimetry, electrophoresis, isoelectric focusing, and immunoassays (e.g., David et al., U.S. Pat. No. 4,376,110) such as immunoblotting (see also Current Protocols in Molecular Biology). For example, in one aspect, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling a DNA probe with biotin such that it can be detected with fluorescently labeled streptavidin.

Western blotting analysis, using an antibody that specifically binds to a polypeptide encoded by an altered CPB2 nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered CPB2 nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to severe disease.

In one aspect of this method, the level or activity of polypeptide encoded by a CPB2 nucleic acid in a test sample is compared with the level or activity of the polypeptide encoded by the CPB2 in a control sample. A level or activity of the polypeptide in the test sample that is higher or lower than the level or amount of the polypeptide in the control sample, such that the difference is statistically significant, is indicative of an alteration in the activity or expression of the polypeptide encoded by the CPB2 nucleic acid, and is diagnostic for a susceptibility to severe disease. In another aspect, both the level or amount and the activity of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to severe disease.

### Therapeutic Agents

In one embodiment of the invention, inhibitors of complement activity are administered to an individual that is classified as lacking a protective allele of CPB2. Usually such individuals have been diagnosed with a disease of interest, e.g. rheumatoid arthritis, NO, paroxysmal nocturnal hemoglobinuria, etc., and are treated prophylactically to prevent the worsening of symptoms, or to alleviate the existing disease condition.

A number of complement inhibitors are known in the art and are undergoing clinical trials, see a review by Emlen et al. (2010) Semin Thromb Hemost. 36(6):660-8. Inhibitors include, without limitation, C5 inhibitors such as pexelizumab (Smith et al. (2010) J Thorac Cardiovasc Surg.); SSL7 protein from Staphylococcus aureus (Laursen et al. (2010) Proc Natl Acad Sci 107(8):3681-6); Factor H-related protein 1 (CFHR-1) (Heinen et al. (2009) Blood. 114(12):2439-47); resveratrol (Issuree et al. (2009) FASEB J. 23(8):2412-24); rEV576 (Soltys et al. (2009) Ann Neurol. 65(1):67-75); eculizumab (Rother et al. (2007) Nat Biotechnol. 25(11):1256-64); as well as inhibitors of C3b (Chen et al. (2010) PNAS 107(41):17621-6); and of B and C2 (Kadam et al. (2010) J. Immunol. 184(12):7116-24). each of which references are herein incorporated by reference.

In some embodiments, the complement inhibitor is a C5 inhibitor. In some embodiments the C5 inhibitor is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

Anti-05 antibodies may be administered daily, semi-weekly, weekly, semi-monthly, monthly, etc., at a dose of from about 0.01 mg, from about 0.1 mg, from about 1 mg, from about 5 mg, from about 10 mg, from about 100 mg or more per kilogram of body weight when administered systemically. Smaller doses may be utilized in localized administration, e.g. in direct administration to ocular nerves, etc. Humanized, chimeric human, or human antibodies are preferred for administering to human patients.

In other embodiments the inhibitor is a drug that inhibits C5 activity, particularly a small molecule drug. Such compounds may be those known in the art, or may be developed using the screening methods described herein.

Determining a therapeutically or prophylactically effective amount of the C5 inhibitor compositions can be done based on animal data using routine computational methods. In one embodiment, the therapeutically or prophylactically effective amount contains between about 0.01 mg and about 1 g of inhibitor, e.g. nucleic acid, protein or peptide, etc., as applicable. In another embodiment, the effective amount contains between about 1 mg and about 100 mg of inhibitor, as applicable. In a further embodiment, the effective amount contains between about 10 mg and about 50 mg of the inhibitor, as applicable.

The method also provide for combination therapy, where the combination may provide for additive or synergistic benefits, by combination with a second agent selected from one or more of the general classes of drugs commonly used in the treatment of the disease. For example, where the disease is rheumatoid arthritis, treatment may include corticosteroids and disease modifying drugs. Corticosteroids have a short onset of action, but many disease modifying drugs take several weeks or months to demonstrate a clinical effect. Alternatively, antigen specific treatmet may be used. These agents include methotrexate, leflunomide (Arava™), etanercept (Enbrel™), infliximab (Remicade™), adalimumab (Humira™), anakinra (Kineret™), rituximab (Rituxan™), CTLA4-Ig (abatacept), antimalarials, gold salts, sulfasalazine, d-penicillamine, cyclosporin A, cyclophosphamide azathioprine; and the like.

Corticosteroids, e.g. prednisone, methylpredisone, prednisolone, solumedrol, etc. have both anti-inflammatory and immunoregulatory activity. They can be given systemically or can be injected locally. Corticosteroids are useful in early disease as temporary adjunctive therapy while waiting for disease modifying agents to exert their effects. Corticosteroids are also useful as chronic adjunctive therapy in patients with severe disease.

The therapeutic compositions may be administered in a single dose, or in multiple doses, usually multiple doses over a period of time, e.g. daily, every-other day, weekly, semi-weekly, monthly etc. for a period of time sufficient to reduce severity of the inflammatory disease, which may comprise 1, 2, 3, 4, 6, 10, or more doses.

Determining a therapeutically or prophylactically effective amount of a therapeutic agent can be done based on animal data using routine computational methods. In one embodiment, the therapeutically or prophylactically effective amount contains between about 0.1 mg and about 1 g of nucleic acid or protein, as applicable. In another embodiment, the effective amount contains between about 1 mg and about 100 mg of nucleic acid or protein, as applicable. In a further embodiment, the effective amount contains between about 10 mg and about 50 mg of the nucleic acid or protein, as applicable. The effective dose will depend at least in part on the route of administration. The agents may be administered orally, in an aerosol spray; by injection, e.g. i.m., s.c., i.p., i.v., etc. In some embodiments, administration by other than i.v. may be preferred. The dose may be from about 0.1 μg/kg patient weight; about 1 μg/kg; about 10 μg/kg; to about 100 μg/kg.

The therapeutic compositions are administered in a pharmaceutically acceptable excipient. The term “pharmaceutically acceptable” refers to an excipient acceptable for use in the pharmaceutical and veterinary arts, which is not toxic or otherwise inacceptable. The concentration of active agent in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

This invention will be better understood by reference to the Examples which follow, but those skilled in the art will readily appreciate that the information detailed is only illustrative of the invention as described more fully in the claims which follow thereafter.

### Experimental

### Plasma Carboxypeptidase B Plays a Central Role in Down-Regulating Inflammatory Responses in Rheumatoid Arthritis

The immune and coagulation systems are both implicated in the pathogenesis of rheumatoid arthritis (RA). However, the interplay between the two systems in RA is unclear. Activated by the coagulation enzyme thrombin, plasma carboxypeptidase B (CPB) plays a pro-coagulant role by removing C-terminal lysines from fibrin and thereby preventing fibrinolysis. Recently, inflammatory mediators, including C5a, bradykinin, and osteopontin (OPN), have been identified as additional CPB substrates. Here we show that CPB plays a central role in down-regulating C5a-mediated inflammatory responses in RA. CPB deficiency exacerbated inflammatory arthritis in a mouse model of RA, and cleavage of C5a by CPB suppressed C5a's ability to recruit immune cells in vivo. Genotyping of two non-synonymous SNPs (G505A and C1040T) in the CPB-encoding gene (CPB2) revealed that RA patients who possess the 1040T allele (encoding Ile325 CPB, the variant with the longer half-life) had a lower risk of developing radiographically severe RA. Ile325 CPB was found to be more effective than Thr325 CPB at abrogating the proinflammatory properties of C5a. Cells from RA synovial joints expressed CPB, whose levels were higher in RA than osteoarthritis (OA) synovial fluid. These findings show that CPB plays a critical role in dampening local, C5a-mediated inflammation and represents a molecular link between inflammation and coagulation in RA.

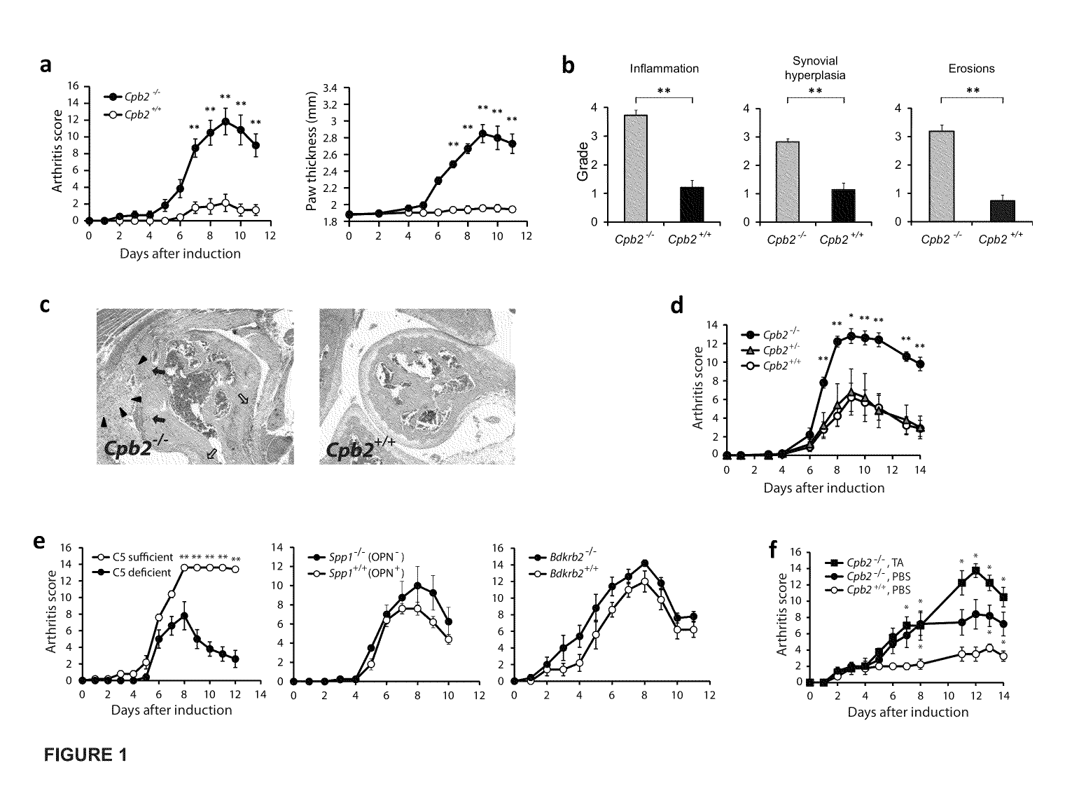

To investigate the role of CPB in inflammatory arthritis, we generated anti-collagen antibody-induced arthritis (CAIA) in mice lacking Cpb2. Seven to ten days after being injected with the anti-collagen antibodies, Cpb2^{-/-} mice exhibited more severe arthritis than Cpb2^{+/+} mice (FIG. 1a). Histologic analysis of joint sections revealed greater erosive damage, synovial hyperplasia, and inflammatory cell infiltration in Cpb2^{-/-} compared to Cpb2^{+/+} mice (FIG. 1 b, c). These findings demonstrate that CPB protects against the development of inflammatory arthritis. To assess the gene-dose effect of Cpb2, we compared the severity of CAIA in Cpb2^{-/-}, Cpb2^{+/-}, and Cpb2^{+/+} mice, and found that Cpb2 heterozygosity is sufficient to protect against the severe CAIA seen in Cpb2^{-/-} mice (FIG. 1d).

To determine which of CPB's substrates is involved in CAIA, we induced CAIA in C5-deficient mice, OPN-deficient mice, and bradykinin B2 receptor-deficient mice. We chose bradykinin B2 receptor-deficient mice over bradykinin B1 receptor-deficient mice because the B2 receptor binds uncleaved bradykinin with greater affinity than CPB-cleaved bradykinin, whereas the B1 receptor preferentially binds CPB-cleaved bradykinin. Compared to controls, C5-deficient mice were resistant to CAIA (FIG. 1e), consistent with previous findings. In contrast, there was no difference in arthritis severity between OPN-deficient mice or bradykinin B2 receptor-deficient mice and their respective controls (FIG. 1e).

By cleaving the C-terminal lysine residues exposed on partially degraded fibrin, CPB decreases the binding of plasminogen to fibrin, thereby suppressing the generation of plasmin; Thus, in Cpb2^{-/-} mice, there will be more plasmin generation in response to fibrin deposition. Plasmin could contribute to the pathogenesis of arthritis by promoting cartilage degradation, either by directly cleaving cartilage proteoglycans or by activating matrix metalloproteinases. To determine whether an increase in plasmin levels accounts for the exacerbation of arthritis in Cpb2^{-/-} mice, we induced CAIA in Cpb2^{-/-} mice and treated them with tranexamic acid (TA), which blocks plasmin generation. TA treatment did not reverse, but instead accentuated, the exacerbation of CAIA in Cpb2^{-/-} mice (FIG. 1f). These findings suggest that the increase in arthritis severity in Cpb2^{-/-} mice is not mediated by plasmin and are consistent with previous findings in which plasmin inhibition increased accumulation of fibrin in the synovium and exacerbated arthritis. These observations likely reflect fibrin's proinflammatory properties.

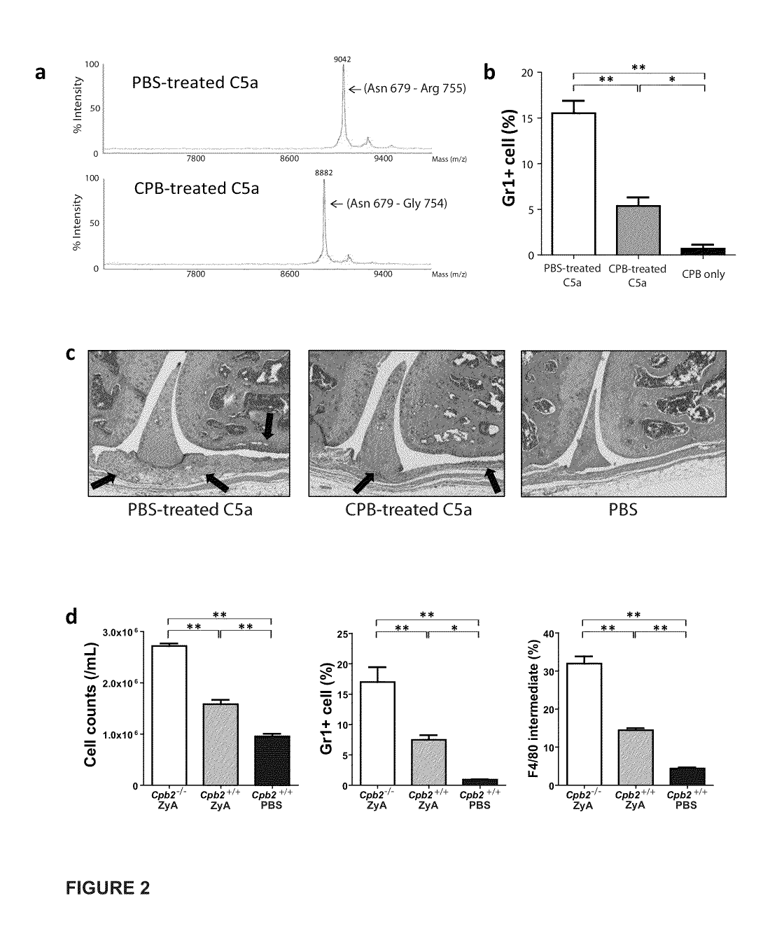

To further explore the interaction between CPB and C5a, we examined the effect of CPB-mediated cleavage on the chemotactic properties of C5a in vivo. Because it is difficult to obtain sufficient mouse synovial fluid for cellular analyses, we initially assessed C5a's ability to recruit inflammatory cells to the mouse peritoneal cavity. We incubated recombinant C5a with CPB in order to generate C5a-desArg (the CPB-cleaved form of C5a, which lacks the C-terminal arginine) and confirmed removal of the C-terminal arginine by mass spectrometry (FIG. 2a). We then injected C5a that had been treated with either CPB or PBS into the peritoneal cavity of wild-type mice, and 5 hours later harvested peritoneal fluid for flow-cytometric analysis. There were fewer neutrophils in peritoneal fluid from mice administered CPB-treated C5a than from those administered PBS-treated C5a (FIG. 2b). CPB-treated C5a also induced less synovial inflammation than PBS-treated C5a when injected into the stifle (knee) joints of mice (FIG. 2c). Furthermore, using the Zymosan A (ZyA)-induced peritonitis model, in which inflammatory cells are recruited to the peritoneum in a C5a-dependent manner, we found that cell recruitment was greater in Cpb2^{-/-} compared to Cpb2^{+/+} mice (FIG. 2d). These data suggest that CPB is anti-inflammatory by cleaving C5a, thus inactivating C5a's chemotactic properties.

We next performed genotyping to determine whether polymorphisms in the CPB2 gene are associated with human RA. Two nonsynonymous single-nucleotide polymorphisms (SNPs) have been reported in the coding region of CPB2: rs3742264 [G505A encoding Ala147Thr] and rs1926447 [C1040T encoding Thr325Ile]. Only the C1040T SNP has known functional consequences, with Ile325 CPB having a two-fold longer half-life and being more effective at inhibiting fibrinolysis than Thr325 CPB. Ile325 CPB has been associated with increased risk of certain thrombotic diseases. We genotyped the two nonsynonymous CPB2 SNPs in an African-American RA cohort (the CLEAR registry) and in age- and gender-matched healthy individuals.

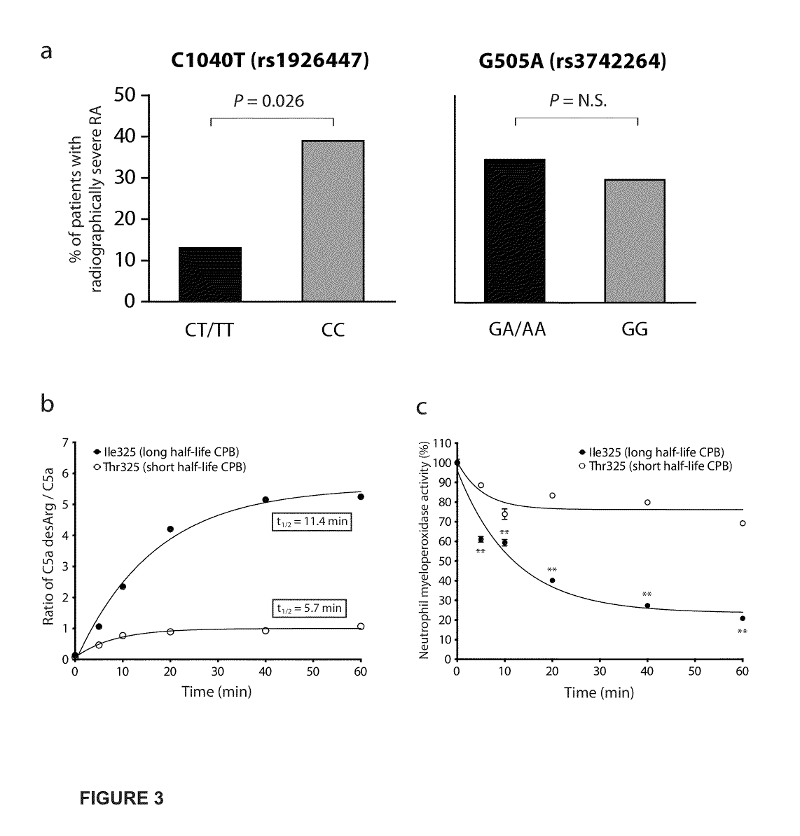

Using our genotyping data, we first asked whether the C1040T SNP (rs1926447) is associated with susceptibility to RA and found there to be no significant association (Table 1). We next asked whether the C1040T SNP is associated with severity of RA, as assessed by radiography of hands and feet and quantified by the modified Sharp/van der Heijde Score (SHS). For this analysis, we used data from the subset of CLEAR RA patients for which the 3-year follow-up SHS were available (n=118), and divided the patients into those with severe radiographic RA (defined as the top tertile of SHS) and those with mild RA (defined as the middle and bottom tertiles of SHS). We then determined how many of the patients with radiographically severe RA were homozygous (CC) for the 1040C allele (encoding short-half-life CPB) and how many were carriers (CT or TT) of the 1040T allele (encoding long-half-life CPB). We found that, compared to 1040C homozygotes, carriers of the 1040T allele had a relative risk reduction of 70% (P=0.026 by chi-square test) for developing radiographically severe RA within 3 years (FIG. 3a, Table 2). There were no significant differences in age, disease duration, DAS28, or other non-radiographic markers of disease activity between 1040C homozygotes and 1040T carriers (Table 3). G505A (rs3742264), the nonsynonymous CPB2 SNP that has no known functional consequence, was not associated with radiographic severity.

To gain insight into the mechanism underlying the genotype association, we compared the ability of the CPB variants encoded by the C1040T SNP to inactivate C5a. We incubated C5a with Thr325 CPB (encoded by 1040C) or Ile325 CPB (encoded by 1040T), and used mass spectrometry to measure the generation of C5a-desArg. By evaluating the ratio of C5a-desArg to intact C5a, we found that the long-half-life Ile325 CPB is more effective than the short-half-life Thr325 CPB at cleaving C5a (FIG. 3c). Furthermore, after 1 hour, C5a treated with Ile325 CPB retained only 21% of its activity, as assessed by its ability to induce neutrophil myeloperoxidase release, whereas C5a treated with Thr325 CPB retained as much as 69% of its activity (FIG. 3d). Thus, the long-half-life Ile325 CPB is three times as effective as the short-half-life Thr325 CPB at neutralizing the proinflammatory mediator C5a, a difference that may provide the molecular basis for the protective effect of the CPB2 1040T allele on radiographic severity in RA.

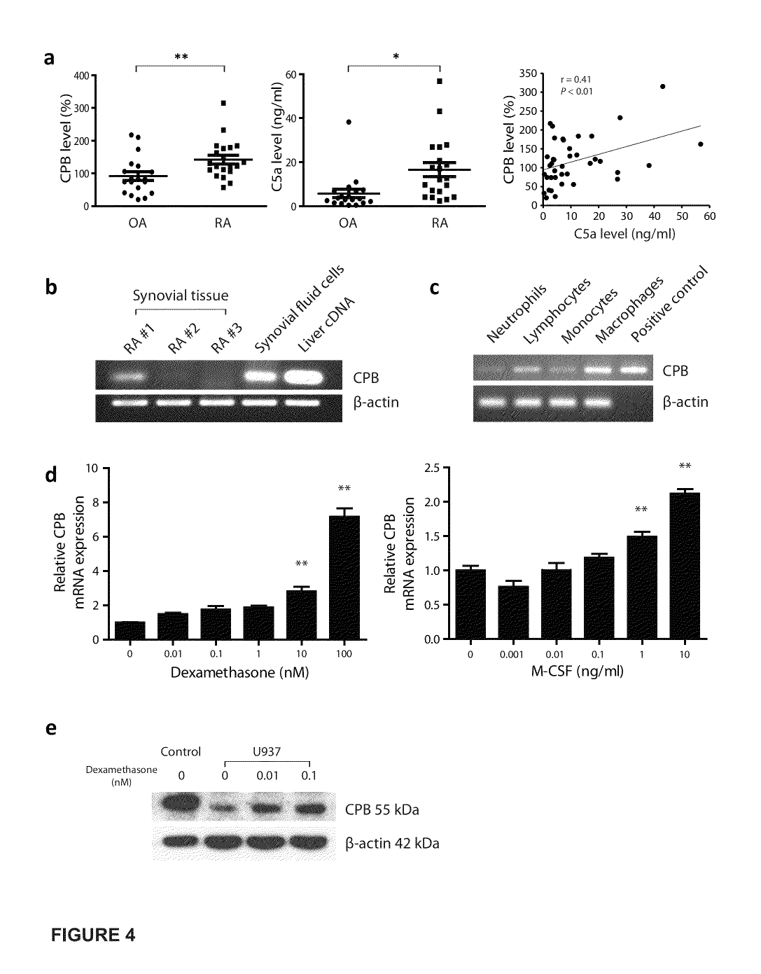

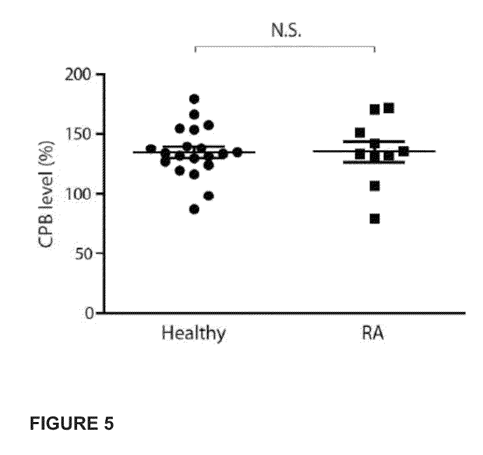

We next measured C5a and CPB protein levels in synovial fluid and plasma from RA patients. Total CPB levels were higher in RA compared to OA synovial fluid (FIG. 4a), consistent with previous findings. Total C5a levels were also higher in RA compared to OA synovial fluid samples, and synovial fluid CPB levels correlated with C5a levels (FIG. 4a). Our data demonstrating an anti-inflammatory role for CPB in murine inflammatory arthritis suggest that the increase in CPB expression in RA occurs as part of an anti-inflammatory feedback mechanism. We did not observe any differences in plasma CPB levels between RA patients and healthy individuals (FIG. 5), suggesting that the increase in CPB levels in synovial fluid in RA is due to local production of CPB.

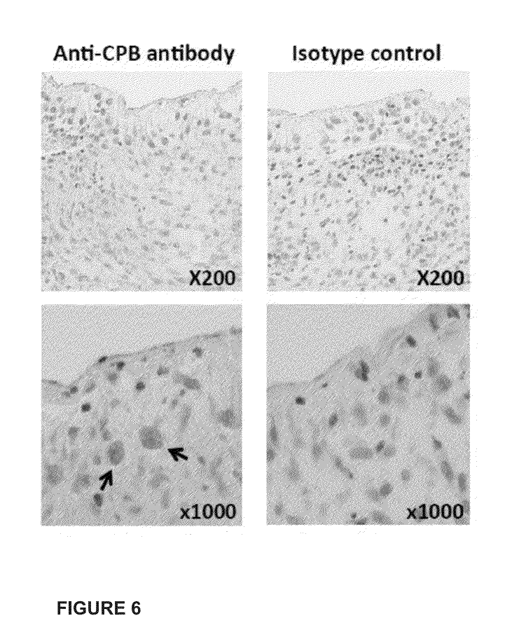

To determine whether CPB is produced locally in the synovial joints in RA, we examined CPB2 mRNA expression in synovial tissue and cells from synovial fluid. Although we recently showed that cultured synoviocytes can produce CPB, assessment of CPB2 expression in situ revealed CPB2 mRNA expression in cells from RA synovial fluid but rarely in RA synovial tissues (FIG. 4b). Furthermore, immunohistochemical analysis showed that CPB protein is present in RA synovial tissues in an interstitial pattern, rarely in the tissue's cells (FIG. 6). Because inflammatory cells in synovial fluid derive from blood, we assessed CPB2 expression in cells obtained from peripheral blood. Macrophages differentiated in vitro from peripheral blood mononuclear cells expressed CPB (FIG. 4c), and the macrophage differentiation factor M-CSF increased CPB2 expression in the monocyte cell line U937 (FIG. 4d). In addition, dexamethasone increased CPB2 mRNA and CPB protein expression in U937 (FIG. 4d, e). Thus, local production by synovial macrophages and fibroblasts can account for the increase in CPB level in RA joints.

Together, our findings demonstrate that CPB, a component of the coagulation system, plays a critical role in down-regulating local inflammation in RA. CPB exerts not only an anti-fibrinolytic effect, but also an anti-inflammatory one. By cleaving C5a, CPB dampens inflammation in the synovial joints and ultimately reduces joint damage in RA.

### Methods

Mouse studies. Cpb2^{-/-} mice were backcrossed >9 generations to the C57BL/6J background at the Jackson Laboratory, and C57BL/6J mice or Cpb2^{+/+} littermates were used as controls. C5-deficient mice, Spp1^{-/-} mice, Bdkrb2^{-/-} mice, and controls were purchased from Jackson Laboratory. Mice were housed at Stanford University and experiments performed under protocols approved by the Stanford University Committee of Animal Research and in accordance with NIH guidelines. CAIA was induced by tail vein injection of 2-8 mg of anti-collagen II antibody (Arthrogen-CIA, Chondrex), followed by i.p. injection of 50 μg of lipopolysaccharide (Sigma) 3 days later. Mice were scored daily for arthritis by using the visual scoring system (see Supplementary methods online) and by measuring paw thickness. For the TA studies, mice received 15 mg of TA (Pfizer) via subcutaneous injection 1 day before the injection of anti-collagen antibody, and then daily until the conclusion of the study, using previously described protocols³¹. Intra-articular and i.p. injection of ZyA or C5a were performed using Cpb2^{-/-} or Cpb2^{+/+} mice as described in the Supplementary Methods.

Human studies. The CLEAR (Consortium for the Longitudinal Evaluations of African Americans with Early Rheumatoid Arthritis) registry. The CLEAR registry enrolled self-declared African Americans who were diagnosed with RA. CLEAR I is a longitudinal cohort of early RA (disease duration of <2 years from the time of symptom onset), and CLEAR II is a cross-sectional cohort of RA of any duration.

Genotyping for CPB2 SNPs rs1926447 and rs3742264 was performed using a custom Infinium iSelect Genotyping Beadchip (Illumina) on DNA samples from the CLEAR I (n=337) and CLEAR II (n=446) registries and from control healthy individuals (n=808), as part of the International MHC and Autoimmunity Genetics Network (IMAGEN). For analysis of associations with RA susceptibility, we used genotyping data from a group of 808 healthy African Americans matched for age and gender, ˜500 persons recruited as part of the CLEAR registry and ˜300 recruited from the Birmingham area (samples were kindly provided by Drs. Robert Kimberly and Jeffrey Edberg). For analysis of associations with radiographic severity, we used genotyping data from RA patients in the CLEAR I registry who were assigned an SHS at the 3-year visit (n=139). Radiographs of the hands and feet were obtained at baseline and at 3 years, and radiographic damage was quantified using the SHS. Clinical data, laboratory data, radiographic data, and CPB2 SNP data were analyzed for female patients only (n=118), because the relatively small number of men who possess the CPB2 SNP rs1926447 minor allele in the CLEAR I registry (n=4) precluded meaningful analysis.

Statistical analyses All statistical data are presented as means±s.e.m except where indicated otherwise. Statistical differences were assessed by Student's t-test for ELISA data and by Mann-Whitney U test for in vivo experiments. For evaluation of the effect of CPB2 SNPs on disease susceptibility or severity, the chi-square test was used. For assessment of the correlation between synovial CPB level and C5a level, the Pearson's correlation test was used.

When multiple comparisons were sought, one-way ANOVA with appropriate post hoc tests was used. n all analyses, P values less than 0.05 were considered significant. Statistical analyses were performed using SPSS for Windows, version 16.0 (SPSS), or GraphPad Prism, version 5.1 (GraphPad).

Human samples Human plasma, synovial fluid, and synovial tissue samples were collected from healthy individuals and from rheumatoid arthritis (RA) and osteoarthritis (OA) patients who met the American College of Rheumatology criteria. All samples were obtained and used under human subjects protocols approved by the Investigational Review Board.

Scoring of murine arthritis. Arthritis in mice was scored according to the following visual scoring system: grade 0, no swelling or erythema; grade 1, mild swelling and erythema or digit inflammation; grade 2, moderate swelling and erythema confined to the region distal to the mid-paw; grade 3, pronounced swelling and erythema with extension to the ankle; grade 4, severe swelling, erythema, and joint rigidity of the ankle, foot, and digits. Each limb was graded with a score of 0-4, with a maximum possible score of 16 for each individual mouse. Paw thickness was determined by measuring the thickness of the hind paws with O— to 10-mm calipers.

Tissue processing. For histologic and immunohistochemical analysis, joint tissue was harvested, decalcified, and embedded with paraffin. Sections of the paraffin-embedded tissue were stained with H&E and scored by a blinded examiner for inflammatory cell infiltrates, erosion of cartilage or bone, and synovial hyperplasia.

Zymosan A-induced peritonitis model. Peritoneal inflammation was induced as previously described. Zymosan A (Sigma) was prepared (2 mg/ml) in sterile PBS and 0.5 ml was i.p. injected into mice. At selected time points, animals were euthanized and peritoneal cells were harvested by lavage with 5 mM EDTA-PBS. Cells were counted with a hemocytometer and stained for granulocytes and macrophages with anti-mouse F4/80 and Gr1 (BD Biosciences). Cells (one million cells per tube) were incubated with purified Fc block (anti-mouse CD16.2/32.2) for 5 min, washed, resuspended in staining buffer and analyzed using a FACScaliber flow cytometer (BD Biosciences).

In vivo chemotaxis assay. 120 μM of C5a (R&D systems) was incubated with 70 nM of activated CPB (American Diagnostica) in Hanks' balanced salt solution (HBSS) for 45 min at room temperature and for 15 min at 3TC. Analysis of full-length C5a (Asn679-Arg755) and CPB-cleaved C5a (Asn679-Gly754) was performed on a PerSeptive Voyager-DE RP Biospectrometry MALDI-TOF (matrix-assisted laser desorption ionization-time of flight) mass spectrometer operating in linear mode using delayed extraction under standard conditions at the Stanford Protein and Nucleic Acid Facility. 2.5 μg of CPB-treated or PBS-treated C5a was injected into mouse peritoneum. Five hours after injection of C5a, peritoneal cells were harvested and analyzed as described above.

Intra-articular injection of C5a. 5 μl of CPB-treated or PBS-treated C5a (0.5 μg/μl) were injected into the mouse stifle joint using a 29-G needle and microsyringe (Hamilton Company). Stifle joints were harvested and processed for H&E staining 72 hours later.

Effect of CPB variants on C5a cleavage and activity. Recombinant human Thr325 CPB and Ile325 CPB were expressed in baby hamster kidney cells and purified as described previously. Each form of CPB (0.2 μM) protein was activated with thrombin (5 nM), thrombomodulin (20 nM), HEPES (20 mM) and CaCl₂ (5 mM) in HBSS at 24° C. for 10 min, after which the thrombin was quenched with PPACK (1 uM). Recombinant human C5a (1 μM) was then hydrolyzed by each form of activated CPB protein (2 nM) at 3TC. Aliquots were removed at 0, 5, 10, 20, 40, and 60 min; and C5a cleavage was stopped by 1 mM EDTA (pH 7.6). To evaluate CPB-mediated cleavage of C5a, we measured levels of total C5a and C5a-desArg in each reaction mix using Exactive (Thermo Fisher) orbitrap mass spectrometer coupled to UPLC (Waters) chromatograph equipped with Poroshell 300SB-C3 75×2 mm column (Agilent). Detection was performed in m/z 600-2000 mass range using electrospray ionization in positive mode. Spectra of multiply charged ions were deconvoluted using ProMass software (ThermoFisher). The intensity ratio of C5a-desArg/C5a was calculated for each time point. To evaluate CPB-mediated inhibition of C5a activity, we measured the release of myeloperoxidase from neutrophils incubated with the CPB-treated C5a. Briefly, neutrophils were prepared, according to a published protocol, from buffy-coat concentrates obtained from the Stanford Blood Bank. Neutrophils (4×10⁶ cells/ml) were resuspended in HBSS with 0.25% bovine serum albumin. 1 ml of neutrophils was treated with 5 μg/ml of cytochalasin B for 5 min at 3° C. 1 μl of each reaction mix of CPB-cleaved C5a (as described above) was added to the neutrophil suspension, incubated for 15 min at 37° C., and centrifuged. MPO released into the supernatant was measured by a commercial MPO activity assay kit (Invitrogen) at A₅₉₀, as recommended.

ELISA for total CPB and total C5a A commercial CPB ELISA kit (Zymutest CPB antigen ELISA kit, Aniara) was used for measurement of CPB protein levels in synovial fluid and plasma. In brief, samples were diluted 1:100. To block non-specific cross-linking by rheumatoid factor, we preincubated synovial fluid samples with 3 μg/ml of HeteroBlock (Omega Biologicals). 200 μl of samples and the standard CPB solution were used to measure total CPB level according to manufacturer's instructions. Synovial C5a level was measured with a commercial C5a ELBA kit (Human C5a ELISA Kit II, BD Biosciences) according to manufacturer's instructions after preincubation with HeteroBlock.

Cell isolation and culture. Human promonocytic U937 were obtained from the American Type Culture Collection. Buffy coat concentrates were obtained from the Stanford Blood Bank. Neutrophils and mononuclear cells were isolated by centrifugation at 400 g for 40 min with Ficoll-Hypaque. Isolated mononuclear cells (1×10⁹ cells) were filtered with a magnetic cell-sorting system (monocyte isolation kit II, Miltenyi Biotec) for selection of magnetically labeled non-monocytic cells. Retained cells were collected as the non-monocytic cell population, and filtered cells as the monocytic cell population. For the generation of macrophages, isolated monocytes were cultured with M-CSF (30 ng/ml, Peprotech) in RPMI-1640 containing 10% FBS for 6 days. Huh-7.5 cells are a human liver hepatoma cell line and were generously provided by Dr. Choongho Lee (Glenn lab, Stanford University Medical School).

RT-PCR and quantitative PCR. Total RNA was isolated from cells or synovial tissue by using Qiagen RNeasy minikits and reverse transcribed into cDNA by using qScript cDNA synthesis kit (Quanta Bioscience). cDNA was amplified by PCR using Top DNA polymerase with universal PCR premix (Bioneer). Primer sequences for standard PCR amplification were as follows. CPB2 forward primer: 5′-CCATGCCAGAGAATGGATCT-3′, CPB2 reverse primer: 5′-ATTCAGGTCTGTTCCGATGC-3′, ACTB forward primer: 5′-CCAACCGCGAGAAGATGA-3′, ACTB reverse primer: 5′-TAGCACAGCCTGGATAGCAA-3′. Liver cDNA (Clontech) was used as a positive control. Quantitative PCR was performed using SYBR Green PCR Kits with the Taqman system (Thermo), and CPB mRNA levels were normalized according to levels of the housekeeping gene hypoxanthine-guanine phosphoribosyl transferase (HPRT). Primer sequences for quantitative PCR analysis were as follows: HPRT forward primer, CAGGCAGTATAATCCAAAGAT; HPRT reverse primer, TCTGGCTTATATCCAACACTTC; CPB2 forward primer, GCCGTGTGTGTACCTG; CPB2 reverse primer; AAAGGTGCGTCAAGTT.

Western Blotting. Following stimulation with dexamethasone (American Regent), U937 or Huh-7.5 cells were lysed with cell lysis buffer (Mammalian Protein Extraction Reagent, Pierce) containing a protease and phosphatase inhibitor cocktail (Fisher Scientific). Protein lysates were separated by sodium dodecyl sulphate-polyacrylamide gel electrophoresis (SDS-PAGE), and western blotting performed using mouse monoclonal anti-CPB antibody (Haematologic Technologies) or mouse monoclonal anti-β-actin antibody (Sigma).

Immunohistochemisty. Paraffin-embedded RA synovial tissue sections were prepared through a series of xylene and alcohol rinses and hydration with H₂O. Endogenous peroxidase activity was quenched with 3% H₂O₂ solution (EMD chemicals), and non-specific binding blocked with serum-free protein block (Dako). The tissue sections were incubated with rabbit polyclonal anti-CPB antibody (Sigma) or normal rabbit IgG (Santa Cruz), and labeled with Envision+Rabbit-HRP polymer (Dako). Staining was developed with DAB+ substrate-chromogen solution (Dako), and counter staining performed with hematoxylin (Dako).

TABLE 1
Nonsynonymous CPB2 SNP case-control analysis

| | rs1926447 | rs3742264 |
|---|---|---|
| | (C1040T) | (G505A) |
| Sample size, cases/controls* | 771/805 | 778/803 |
| Genotype | | |
| Cases | | |
| AA | 598 | 271 |
| AB | 157 | 391 |
| BB | 16 | 116 |
| Controls | | |
| AA | 599 | 301 |
| AB | 186 | 380 |
| BB | 20 | 122 |
| Minor Allele frequency | | |
| Cases | 0.12 | 0.40 |
| Controls | 0.14 | 0.39 |
| Allelic χ2 P value | 0.14 | 0.50 |
| Genotypic association | | |
| (AA vs. AB + BB) | | |
| Odds ratio | 0.84 | 1.12 |
| 95% confidence interval | 0.67-1.06 | 0.91-1.38 |
| χ2 | 2.14 | 1.20 |
| P | 0.14 | 0.27 |

*Indicates the number of samples successfully genotyped. A = major allele for each SNP, for rs1926447 the major allele is C and for rs3742264 G. B = minor allele for each SNP, for rs1926447 the minor allele is T and for rs3742264 A.

TABLE 2
CPB2 SNP rs1926447 genotype frequency in
RA stratified by radiographic severity

| | | | Odd | Relative | |
|---|---|---|---|---|---|
| | | | Ratio | Risk | |
| | CT/TT | CC | (95% CI) | (95% CI) | P |
| Severe*, n (%) | 3 (13.0) | 37 (38.9) | 0.24 | 29.3% | 0.026 |
| | | | (0.07-0.85) | (9.2-92.6) | |
| Mild^{#}, n (%) | 20 (87.0) | 58 (61.1) | | | |

*Defined as the top tertile of radiographic severity as measured by modified Sharp/van der Heijde score (SHS).
^{#}Defined as the middle and bottom tertiles of radiographic severity as measured by SHS.

TABLE 3
Clinical, laboratory, and radiologic characteristics of the patients in
the CLEAR I registry stratified by CPB2 SNP rs1926447

| | CT/TT | | CC | | P |
|---|---|---|---|---|---|
| Number (female %) | 23 | (100) | 95 | (100) | |
| Age, years | 55.4 | (± 10.6) | 59.8 | (± 13.6) | 0.095 |
| disease duration (months) | 103.4 | (± 12.8) | 98.8 | (± 16.4) | 0.148 |
| Smoking (%) | 10 | (43.5) | 54 | (56.8) | 0.263 |
| Anti-CCP positive, n (%) | 15 | (65.2) | 62 | (65.3) | 0.798 |
| RF positive, n (%) | 19 | (82.6) | 69 | (72.6) | 0.721 |
| HLA-DRB1 shared epitope | 11 | (47.8) | 46 | (48.4) | 0.960 |
| present, n (%) | | | | | |
| CRP (mg/L), Year 0 | 9.9 | (± 22.5) | 15.2 | (± 33.4) | 0.374 |
| Tender joint count (0-38) | 7.2 | (± 7.3) | 8.7 | (± 8.9) | 0.409 |
| Swollen joint count (0-42) | 4.9 | (± 6.2) | 4.8 | (± 6.4) | 0.968 |
| DAS28, Year 0 | 3.7 | (± 1.4) | 4.0 | (± 1.4) | 0.371 |
| HAQ, Year 0 | 1.7 | (± 0.9) | 1.8 | (± 0.9) | 0.942 |
| Treatment with | 21 | (91.3) | 80 | (84.2) | 0.323 |
| DMARDs, n (%) | | | | | |
| SHS, Year 0 | 0.3 | (± 0.8) | 2.3 | (± 5.3) | 0.001 |
| SHS, Year 3 | 1.8 | (± 3.9) | 5.9 | (± 12.0) | 0.007 |
| Erosion score, Year 0 | 0.3 | (± 0.8) | 1.0 | (± 2.4) | 0.035 |
| Erosion score, Year 3 | 0.6 | (± 1.1) | 2.6 | (± 6.5) | 0.005 |
| JSN score, Year 0 | 0.0 | (± 0.0) | 1.3 | (± 3.5) | <0.001 |
| JSN score, Year 3 | 1.2 | (± 3.2) | 3.2 | (± 6.3) | 0.033 |

Except where indicated otherwise, values are the mean ± s.d.
CCP: Cyclic Citrullinated Peptide Antibody;
RF: Rheumatoid Factor;
DMARD: disease-modifying anti-rheumatic drug;
SHS: modified Sharp/van der Heijde score;
JSN: joint-space narrowing;
HAQ: Health Assessment Questionnaire;
DAS28: disease activity score based on 28 joints;
CRP: C-reactive protein;
HLA-DRB1 shared epitope alleles were *0101, *0102, *0401, *0404, *0405, *0413, *1001 and *1402.

1. A method of diagnosing a susceptibility to development or increased severity of a complement-associated disease in an individual, comprising determining the presence or absence of a polymorphic allele in a biological sample from said individual, wherein the polymorphic allele is genetically linked to carboxypeptidase B (CPB2) locus.

2. The method of claim 1, wherein the polymorphic allele comprises a single nucleotide polymorphism.

3. The method of claim 2, wherein the single nucleotide polymorphism is within intron or exon of the CPB2 locus.

4. The method of claim 1, wherein the polymorphic allele encodes a variant of CPB2 having increased stability, wherein said variant having increased stability is protective of development of severe disease.

5. The method of claim 4, wherein the variant having increased stability is CPB2 Thr325Ile.

6. The method of claim 1, wherein the complement-associated disease is rheumatoid arthritis.

7. The method of claim 4, wherein the individual has been diagnosed with rheumatoid arthritis.

8. The method of claim 1, wherein said biological sample is a genetic sample.

9. The method of claim 8, wherein the genetic sample comprises mRNA or a cDNA derived therefrom.

10. The method of claim 9, wherein the polymorphic allele is detected by determining the presence of an SNP selected from rs1928447 and rs1409433.

11. The method of claim 1, wherein the biological sample is a protein sample.

12. The method of claim 1, further comprising the step of administering to an individual determined to be susceptible to development or increased severity of a complement-associated disease an inhibitor of complement activity.

13. The method of claim 12, wherein the inhibitor is an inhibitor of complement C5.

14. The method of claim 13, wherein the inhibitor is an antibody.

15. A kit for assessing susceptibility to development or increased severity of a complement-associated disease in an individual, the kit comprising reagents for selectively determining the presence or absence of at least one polymorphic allele in a biological sample from said individual, wherein the polymorphic allele is genetically linked to carboxypeptidase B (CPB2) locus.

16. The kit according to claim 15, comprising probes that specifically bind to at least one SNP selected from rs1926447 and rs1409433.

17. The kit according to claim 15, comprises reagents for determining the presence of CPB2 Thr325Ile protein.
